(19) United States
(12) Patent Application Publication
(10) Pub. No.: US 2026/0345727 A1
(43) Pub. Date: Nov. 5, 2026

(54) PEPTIDE VETERINARY COMPOSITION FOR IMMUNOTHERAPY

(71) Applicant: INSTITUTO POLITECNICO NACIONAL, Ciudad De México, (MX)

(72) Inventor: Sonia Mayra Perez Tapia, Ciudad De México (MX)

(21) Appl. No.: 19/118,561

(22) PCT Filed: Oct. 6, 2022
(86) PCT No.: PCT/IB2022/059525

§ 371 Date: Apr. 4, 2025

(51) Int. Cl. A61K 38/17; A61K 9/00; A61K 38/22; A61K 38/36; A61K 38/42; A61P 31/20

(52) U.S. Cl. CPC A61K 38/1709; A61K 9/0019; A61K 38/1719; A61K 38/1725; A61K 38/2292; A61K 38/363; A61K 38/42; A61P 31/20

(57) ABSTRACT

The present invention relates to a veterinary composition for use in the treatment, control and prevention of a disease in an animal, wherein the veterinary composition comprises a peptide mixture, and at least one veterinary acceptable vehicle or adjuvant; wherein the peptide mixture further comprises a full human Ubiquitin (Ub) monomer, a variant of the human Ubiquitin monomer lacking two terminal glycine (Ub(-GG)) and at least one low molecular weight human peptide.

### TECHNICAL SECTOR

The present invention relates to veterinary medicine, more particularly it relates to a veterinary composition with immunomodulatory effects.

### BACKGROUND OF THE INVENTION

The global veterinary care market is projected to reach USD 114,000 million by 2028, with a compound annual growth rate (CAGR) of 5.4% during the forecast period, according to a recent report by Grand Vies Research, Inc. Additionally, the increase in the number of government initiatives to ensure public health will likely drive the adoption of better veterinary care around the world.

Proper management of veterinary health through prevention, detection, and appropriate treatment is a fundamental element in preventing zoonoses. Approximately 60% of all infectious diseases in humans and 75% of emerging infectious diseases are zoonotic, meaning they are transmitted by animals. Recent or re-emerging zoonoses include Ebola, avian flu, Middle East Respiratory Syndrome (MERS), Nipah virus, Rift Valley fever, Severe Acute Respiratory Syndrome (SARS), West Nile virus, Zika virus, and now, the coronavirus causing COVID-19.

Currently, some researchers have focused on the development of new treatments to prevent veterinary diseases through the development of drugs that stimulate the immune system of animals during the period of infection. For example, U.S. Pat. No. 4,748,018 describes the administration of avian antibodies to mammals to provide protection against oral and gastrointestinal infections. Specifically, the document describes a method of passive immunization, comprising: (a) feeding a mammal with a material having an enhanced antibody titer against an antigen, obtained from the egg of a domesticated bird immunized against that antigen, until the mammal develops substantial tolerance to the antibodies; and (b) administering to the mammal an antibody obtained from a domesticated bird immunized against that antigen. In other words, prior to administration to the mammal of antibodies obtained from domesticated birds, the mammal is fed a material derived from eggs of birds immunized against the antigen. The need to feed the mammalian material from immunized eggs prior to the administration of the avian antibodies obviously means that the method has very limited utility against acute infections and diseases. Furthermore, it is difficult, if not impossible, to apply the method to neonates.

On the other hand, patent document EP0930316 describes oral products for the treatment of dogs and calves, comprising specific antibodies against bovine rotavirus, bovine coronavirus, enterotoxigenic strains of Escherichia coli, and/or canine parvovirus, obtained from immunized cows (colostrum) or hens (egg yolk, combined with stabilized live lactic acid bacteria). However, is generally undesirable to treat mammalian pathogenic infections with avian antibodies, since mammalian immune systems are likely to respond negatively to these large molecules. Furthermore, since mammalian immune systems do not recognize avian antibodies as useful due to their ability to recognize certain pathogens or the specificities of avian antibodies for the antigens of such pathogens, avian antibodies do not even induce the desired immune responses in mammals.

Consequently, efforts have been made to overcome the limitations of different veterinary products currently used in immunotherapy by developing a veterinary composition of peptides with immunomodulatory effects that does not exhibit variability in peptide content based on cellular origin and manufacturing method. Furthermore, these components have a higher affinity for binding to specific receptors in different mammals, suggesting a greater immunomodulatory effect.

### OBJECTS OF THE INVENTION

Considering the deficiencies of the prior art, it is an object of the present invention to provide a veterinary peptide composition with immunomodulatory effects that does not present variability in peptide content according to its cellular origin and method of manufacture and that is useful for the comprehensive regulation of the immune system.

It is also another object of the present invention to provide a veterinary peptide composition that stimulates the immune system in an animal.

Another object of the present invention relates to a veterinary composition of peptides that does not exhibit variability in its content.

These and other objects are achieved by a veterinary peptide composition with immunomodulatory effects in accordance with the present invention.

### BRIEF DESCRIPTION OF THE INVENTION

To this end, the first aspect of the present invention relates to a veterinary composition, comprising a peptide mixture at a concentration between 33 and 84% (w/v), and at least one veterinary-acceptable vehicle or adjuvant at a concentration between 16 and 67% (w/v); wherein the peptide mixture comprises a complete monomer of human Ubiquitin (Ub), a variant of the human Ubiquitin monomer lacking two terminal glycine (Ub(-GG)), and human low molecular weight peptides.

A second aspect of the present invention relates to a veterinary composition for use in the treatment, control, and prevention of a disease in an animal, wherein the veterinary composition comprises a peptide mixture at a concentration between 33 and 84% (w/v), and at least one veterinary-acceptable adjuvant or vehicle at a concentration between 16 and 67% (w/v); wherein the peptide mixture includes a complete monomer of human Ubiquitin (Ub), a variant of the human Ubiquitin monomer lacking two terminal glycine (Ub(-GG)), and low molecular weight human peptides.

A third aspect of the present invention relates to the use of a veterinary composition for the manufacture of a drug for the treatment, control, and prevention of a disease in an animal. The veterinary composition comprises a peptide mixture at a concentration between 33 and 84% (w/v), and at least one veterinary-acceptable adjuvant or vehicle at a concentration between 16 and 67% (w/v); wherein the peptide mixture includes a complete monomer of human Ubiquitin (Ub), a variant of the human Ubiquitin monomer lacking two terminal glycine (Ub(-GG)), and low molecular weight human peptides.

### BRIEF DESCRIPTION OF THE FIGURES

The novel aspects considered characteristic of the present invention will be particularly specified in the appended claims. However, some embodiments, features, and certain objects and advantages thereof will be better understood in the detailed description when read in connection with the accompanying drawings, in which:

FIG. 1 illustrates the value of the dissociation constant (Kd) at 37 °C for the binding of Ub (1A) and Ub(-GG) ubiquitins (1B) to the CXCR4 receptor of human, dog, mouse, rabbit, and pig, wherein **p<0.01M; ***p<0.001; N.S.=Not Significant.

FIG. 2 illustrates the percentage of survival in dogs infected with canine parvovirus, treated with CT+V (Conventional Treatment+Vehicle) and CT+C (Conventional Treatment+Veterinary Peptide Composition).

FIG. 3 illustrates the clinical score in dogs infected with canine parvovirus treated with CT+V (Conventional Treatment+Vehicle) over time per dog, wherein the arrow indicates that high values (score of 1 to 41) correlate with the worsening clinical condition of the dog.

FIG. 4 illustrates the clinical score in dogs infected with canine parvovirus treated with CT+C (Conventional Treatment+Veterinary Peptide Composition) over time per dog, wherein the arrow indicates that high values (score of 1 to 41) correlate with worsening clinical condition of the dog.

FIG. 5 illustrates the concentration of circulating neutrophils in dogs infected with canine parvovirus, treated with CT+V (Conventional Treatment+Vehicle) and CT+C (Conventional Treatment+Veterinary Peptide Composition), wherein A is the concentration of circulating neutrophils in surviving dogs on day 2 (critical day); B is the concentration of circulating neutrophils in surviving dogs on day 4 (recovery day); C is the concentration of circulating neutrophils in the CT+V group; and D is the concentration of circulating neutrophils in the CT+C group. Each point represents one dog. *P≤0.05, **P≤0.01.

FIG. 6 illustrates the concentration of circulating lymphocytes in dogs infected with canine parvovirus, treated with CT+V (Conventional Treatment+vehicle) and CT+C (Conventional Treatment+Veterinary Peptide Composition), wherein A is the concentration of circulating lymphocytes in surviving dogs on day 2 (critical day); B is the concentration of circulating lymphocytes in surviving dogs on day 4 (recovery day); C is the concentration of circulating lymphocytes in the CT+V group; and D is the concentration of circulating lymphocytes in the CT+C group. Each point represents a dog. *P≤0.05, **P≤0.01.

FIG. 7 illustrates the plasma concentration of neurotransmitters in dogs infected with canine parvovirus, treated with CT+V (Conventional Treatment+vehicle) and CT+C (Conventional Treatment+Veterinary Peptide Composition), wherein A is the plasma concentration of norepinephrine; B is the plasma concentration of epinephrine; and C is the plasma concentration of serotonin. Each point represents one dog. Median±5-95% confidence interval. *P≤0.05.

### DESCRIPTION OF A MODE OF CARRYING OUT THE INVENTION

A veterinary peptide composition has been found to be useful in immunotherapy for the treatment of diseases related to immunodeficiencies, auto immunities, and/or allergies. Additionally, the disease may be related to a viral, bacterial, or parasitic infection.

For this purpose, the first aspect of the present invention relates to a veterinary composition comprising a peptide mixture at a concentration between 33 and 84% (w/v), and at least one veterinary-acceptable adjuvant or vehicle at a concentration of between 16 and 67% (w/v); wherein the peptide mixture includes a complete monomer of human Ubiquitin (Ub), a variant of the human Ubiquitin monomer lacking two terminal glycine (Ub(-GG)), and low molecular weight human peptides.

A second aspect of the present invention relates to a veterinary composition for use in the treatment, control, and prevention of a disease in an animal; wherein the veterinary composition comprises a mixture of peptides at a concentration between 33 and 84% (w/v) and at least one veterinary-acceptable adjuvant or vehicle at a concentration between 16 and 67% (w/v); wherein the peptide mixture includes a complete monomer of human Ubiquitin (Ub), a variant of the human Ubiquitin monomer lacking two terminal glycine (Ub(-GG)), and low molecular weight human peptides.

A third aspect of the present invention relates to the use of a veterinary composition for the manufacture of a medicament for the treatment, control, and prevention of a disease in an animal, wherein the veterinary composition comprises a mixture of peptides at a concentration between 33 and 84% (w/v) and at least one veterinary-acceptable adjuvant or vehicle at a concentration between 16 and 67% (w/v); wherein the peptide mixture includes a complete monomer of human Ubiquitin (Ub), a variant of the human Ubiquitin monomer lacking two terminal glycine (Ub(-GG)), and low molecular weight human peptides.

Preferably, the animal is a domestic, exotic, or industrially relevant animal.

Preferably, the veterinary composition is adapted to be administrable to an animal by topical, dermal, transdermal, transmucosal, transmucosal, epidermal, parenteral, gastrointestinal, oral, oropharyngeal, nasopharyngeal and pulmonary routes, including transbronchial and transalveolar.

Preferably, the veterinary-acceptable adjuvant is a pharmaceutical, and/or immunogenic, and/or immunostimulant, and/or immunotherapeutic agent. For example, the veterinary composition of peptides with immunomodulatory effects can be administered concurrently with cytokines or antibodies.

Preferably, the vehicle is a physiologically acceptable carrier.

Preferably, the disease is related to immunodeficiencies, autoimmunities, allergies, or a combination thereof.

Preferably, the disease is related to a viral, bacterial, parasitic infection, or a combination thereof.

Preferably, the disease is caused by a virus. More preferably, canine parvovirus type 2 (CPV-2).

Preferably, the veterinary peptide composition promotes the decrease of neutrophil and lymphocyte counts in the critical stage of a disease in an animal, reducing plasma cortisol levels and neurotransmitter changes.

Preferably, the immune response is triggered following the binding of any of the components present in the veterinary peptide composition, more preferably the complete monomer of human Ubiquitin (Ub), and/or the variant of the human Ubiquitin monomer lacking two terminal glycine (Ub(-GG)), to the chemokine 4 receptor (Motive C-X-C) CXCR4 of an animal.

The complete Ub monomer preferably has a molecular mass of about 8.56 kDa. Likewise, the monomer variant of Ub(-GG) preferably has a molecular mass of about 8.45 kDa. The Ub monomer is a naturally occurring serum globular protein of small size (76 amino acids) that lacks post-translational modifications and possesses high thermal, structural, and proteolytic stability.

The complete Ub monomer is selected from the amino acid sequence SEQ ID NO:1. In addition, the Ub(-GG) monomer variant is selected from the amino acid sequence SEQ ID NO:2.

Preferably, the veterinary peptide composition comprises from 0.168 to 0.268% (m/m) of the full-length Ub monomer and the Ub(-GG) monomer variant. The remainder of the veterinary peptide composition consists of minority peptide fragments. More preferably, the average content of the Ub(-GG) monomer variant in the veterinary peptide composition with immunomodulatory effects is in a 2:1 ratio with respect to the average content of the full Ub monomer.

In a preferred embodiment of the present invention, the veterinary peptide composition additionally comprises at least one low molecular weight human peptide Anquirin-1, Protein 4.1, Hemoglobin Alpha Subunit, Hemoglobin Beta Subunit, Hemoglobin Delta Subunit, Complement Protein C3, Calpastatin, Alpha-Synuclein, Fibrinogen Alpha Chain, Cytoplasmic Actin 1, Thymosin Beta-4, Thymosin Beta-10, or Zyxin.

For purposes of the present invention, the term “low molecular weight human peptide” means a continuous segment of a protein, which has a molecular mass of less than 10 kDa.

Preferably, the veterinary peptide composition comprises 0.644 to 0.924% (m/m) of at least one fragment of Anquirin-1. Likewise, in a preferred manner, the veterinary peptide composition comprises 0.397 to 0.637% (m/m) of at least one fragment of Protein 4.1. Likewise, in a preferred manner, the veterinary peptide composition comprises 0.351 to 0.571% (m/m) of at least one Hemoglobin Alpha Subunit fragment. Likewise, in a preferred manner, the veterinary peptide composition comprises 0.367 to 0.547% (m/m) of at least one Hemoglobin Beta Subunit and/or Hemoglobin Delta Subunit fragment. Likewise, in a preferred manner, the veterinary peptide composition comprises from 0.303 to 0.583% (m/m) of at least one fragment of Complement C3 protein. Likewise, in a preferred manner, the veterinary peptide composition comprises 0.319 to 0.519% (m/m) of at least one fragment of Calpastatin. Likewise, in a preferred manner, the veterinary peptide composition comprises 0.198 to 0.298% (m/m) of at least one fragment of Alpha-Synuclein. Likewise, in a preferred manner, the veterinary peptide composition comprises 0.151 to 0.311% (m/m) of at least one Fibrinogen Alpha-Chain fragment. Also, preferably, the veterinary peptide composition comprises 0.163 to 0.283% (m/m) of at least one Cytoplasmic Actin fragment. Also preferably, the veterinary peptide composition comprises 0.12 to 0.2% (m/m) of at least one fragment of Thymosin Beta-4 and/or Thymosin Beta-10. Also preferably, the veterinary peptide composition comprises 0.108 to 0.208% (m/m) of at least one Zyxin fragment.

In a preferred embodiment of the present invention, each low molecular weight human peptide is selected from the human Ankyrin-1 protein, which is preferably selected from ANK1_HUMAN (UniProtKB accession number P16157) or Q6PK32_HUMAN (UniProtKB accession number Q6PK32). More preferably, each low molecular weight human peptide corresponds to a fragment of the amino acid sequence SEQ ID NO:3 or SEQ ID NO:4.

In another preferred embodiment of the present invention, each low molecular weight human peptide is selected from the human Protein 4.1, which is preferably selected from 41_HUMAN (UniProtKB accession number P11171). More preferably, each low molecular weight human peptide corresponds to a fragment of the amino acid sequence SEQ ID NO:5.

In another preferred embodiment of the present invention, each low molecular weight human peptide is selected from the human Hemoglobin Alpha Subunit, which is preferably selected from HBA_HUMAN (UniProtKB accession number P69905) or G3V1 N2_HUMAN (UniProt accession number G3V1 N2). More preferably, each low molecular weight human peptide corresponds to a fragment of the amino acid sequence SEQ ID NO:6 or SEQ ID NO:7.

In another preferred embodiment of the present invention, each low molecular weight human peptide is selected from the human Hemoglobin Beta Subunit, which is preferably selected from HBB_HUMAN (UniProtKB accession number P68871). More preferably, each low molecular weight human peptide corresponds to a fragment of the amino acid sequence SEQ ID NO:8.

In another preferred embodiment of the present invention, each low molecular weight human peptide is selected from the human Hemoglobin Delta Subunit, which is preferably selected from E9PEW8_HUMAN (UniProtKB accession number E9PEW8). More preferably, each low molecular weight human peptide corresponds to a fragment of the amino acid sequence SEQ ID NO:9.

In another preferred embodiment of the present invention, each low molecular weight human peptide is selected from the human Complement C3 Protein, which is preferably selected from CO3_HUMAN (UniProtKB accession number P01024). More preferably, each low molecular weight human peptide corresponds to a fragment of the amino acid sequence SEQ ID NO:10.

In another preferred embodiment of the present invention, each low molecular weight human peptide is selected from the human Calpastatin protein, preferably selected from ICAL_HUMAN (UniProtKB accession number P20810) or AOAOAOMR45_HUMAN (UniProtKB accession number AOAOAOMR45), or D6RC54_HUMAN (UniProtKB accession number D6RC54). More preferably, each low molecular weight human peptide corresponds to a fragment of the amino acid sequence SEQ ID NO:11, SEQ ID NO:12, or SEQ ID NO:13.

In another preferred embodiment of the present invention, each low molecular weight human peptide is selected from the human Alpha-Synuclein protein, preferably selected from SYUA_HUMAN (UniProtKB accession number P37840) or H6UYS7_HUMAN (UniProtKB accession number H6UYS7). More preferably, each low molecular weight human peptide corresponds to a fragment of the amino acid sequence SEQ ID NO:14 or SEQ ID NO:15.

In another preferred embodiment of the present invention, each low molecular weight human peptide is selected from the human Fibrinogen Alpha Chain, preferably selected from FIBA_HUMAN (UniProtKB accession number P02671). More preferably, each low molecular weight human peptide corresponds to a fragment of the amino acid sequence SEQ ID NO:16.

In another preferred embodiment of the present invention, each low molecular weight human peptide is selected from the human Actin, Cytoplasmic 1 protein, preferably selected from ACTB_HUMAN (UniProtKB accession number P60709), I3L3R2_HUMAN (UniProtKB accession number I3L3R2), or POTEE_HUMAN (UniProtKB accession number Q6S8J3). More preferably, each low molecular weight human peptide corresponds to a fragment of the amino acid sequence SEQ ID NO:17, SEQ ID NO:18, or SEQ ID NO:19.

In another preferred embodiment of the present invention, each low molecular weight human peptide is selected from the human Thymosin Beta-4 protein, preferably selected from TYB4_HUMAN (UniProtKB accession number P62328). More preferably, each low molecular weight human peptide corresponds to a fragment of the amino acid sequence SEQ ID NO:20.

In another preferred embodiment of the present invention, each low molecular weight human peptide is selected from the human Thymosin Beta-10 protein, preferably selected from TYB10_HUMAN (UniProtKB accession number P63313). More preferably, each low molecular weight human peptide corresponds to a fragment of the amino acid sequence SEQ ID NO:21.

In another preferred embodiment of the present invention, each low molecular weight human peptide is selected from the human Zyxin protein, preferably selected from ZYX_HUMAN (UniProtKB accession number Q15942). More preferably, each low molecular weight human peptide corresponds to a fragment of the amino acid sequence SEQ ID NO:22.

The immunologically effective doses and method of administration of the immunomodulatory peptide veterinary composition may vary depending on the animal, what condition is to be treated, and other factors apparent to one skilled in the art. Factors to be considered include but are not limited to: the route of administration, the number of doses, and whether the veterinary peptide composition with immunomodulatory effects will be administered along with other veterinary-acceptable vehicles or adjuvants. Such factors are known in the art and it is within the knowledge of those skilled in the art to make such determinations. A suitable dosing range is one that provides the desired modulation of the immune response. Useful dosage ranges of the veterinary peptide composition with immunomodulatory effects may be, for example, from about any of the following: 0.1 to 100 μg/kg, 0.1 to 50 μg/kg, 0.1 to 25 μg/kg, 0.1 to 10 μg/kg, 1 to 500 μg/kg, 100 to 400 μg/kg, 200 to 300 μg/kg, 1 to 100 μg/kg, 100 to 200 μg/kg, 300 to 400 μg/kg, 400 to 500 μg/kg. Alternatively, the doses can be approximately any of the following: 0.1 μg, 0.25 μg, 0.5 μg, 1.0 μg, 2.0 μg, 5.0 μg, 10 μg, 25 μg, 50 μg, 75 μg, 100 μg. Accordingly, the dose ranges maybe those with a lower limit of any of the following: 0.1 μg, 0.25 μg, 0.5 μg, and 1.0 μg; and with an upper limit of approximately any of the following: 25 μg, 50 μg, and 100 μg. The absolute amount administered to each animal depends on pharmacological properties such as bioavailability, clearance rate, and route of administration.

The effective amount and administration method of the veterinary composition may vary depending on the animal, the stage of the disease, and other factors apparent to one skilled in the art. The route(s) of administration useful in a particular application are evident to one skilled in the art. Administration routes include but are not limited to topical, dermal, transdermal, transmucosal, epidermal, parenteral, gastrointestinal, oral, oropharyngeal, nasopharyngeal, and pulmonary, including transbronchial and transalveolar. An appropriate dosage range is one that provides sufficient veterinary peptide composition with immunomodulatory effects to achieve a preventive or therapeutic effect. The absolute amount administered to each patient depends on pharmacological properties such as bioavailability, clearance rate, and route of administration.

The present invention will be better understood from the following examples, which are provided solely for illustrative purposes to enable a full understanding of the preferred embodiments of the present invention, without implying that there are no other embodiments not illustrated that can be carried out based on the detailed description above.

### EXAMPLES

### Example 1. In Silico Analysis of CXCR4 from Different Animals Interacting with Human Ub and Ub(-GG) Ubiquitin

In the present example, an in silico analysis of the veterinary peptide composition in accordance with the present invention is presented. The purpose of the assay was to predict the molecular coupling (docking) of the two major components in the veterinary composition, the complete human Ub Ubiquitin monomer (SEQ ID NO.: 1) and the variant of the human Ubiquitin monomer lacking two terminal Ub(-GG) glycine (SEQ ID NO.: 2) to bind to an animal's CXCR4 Chemokine Receptor 4 (C-X-C motif) CXCR4. The binding and affinity between these molecules are hypothesized to trigger an immune response in mammalian immune response in mammals used as companion animals and/or of commercial interest.

The crystallized structure of human CXCR4 ID. 4RWS (PDB DOI: 10.2210/pdb4RWS/pdb) was edited with Biovia Discovery Visualizer Studio software (BDVS, Discovery Studio Modeling Environment, 2017; Dassault Systemes) to remove accessory proteins (vMIP2 and lysozyme T4). In addition, intramolecular loops of human CXCR4 were recovered using the Maestro v12.5 Protein Preparation Wizard tool (Schrödinger Suite, Protein Preparation Wizard, 2021-1; Schrödinger). In addition, edited human CXCR4 was used to model dog CXCR4 (Accession number Q3LSL6, UNIPROT); mouse CXCR4 (Accession number P70658, UNIPROT); Rabbit CXCR4 (Accession number G1SGF1, UNIPROT); and pig CXCR4 (Accession number Q764M9, UNIPROT) by homology in Modeller v9. The model with the lowest DOPE (Discrete Optimized Protein Energy) score was chosen among the five candidate models.

The crystallized structure of 6KDU (PDB DOI: 10.2210/pdb6KDU/pdb) was used as a model after editing with BDVS to remove accessory molecules (adenine 5-phosphoribose, Zn⁺², and Mg⁺²) for the purpose of Ub and Ub(-GG) homology modeling. The model with the lowest DOPE score was chosen among the candidates for Ub and Ub(-GG).

To couple the CXCR4 receptor of the different animals to human Ub or Ub(-GG), a masking template generated in PyMOL v2.0 (The PyMOL Molecular Graphics System version 2.0; Schrödinger) was used to target the interaction specifically in the extracellular domain of CXCR4. The 10 large clusters obtained in each receptor were analyzed to identify the dissociation constant at 37 °C with the PRODIGY (prediction protein binding energy) server.

From the results obtained, it is exhibited that the Kd at 37 °C between dog CXCR4 and human Ub was 13.5 nM; this value is much lower than the Kd at 37 °C reported for human CXCR4 binding to human Ub (84 nM) by experimental assays. However, this could be due to the inherent bias of the in silico assay compared to previously reported experimental data. On the other hand, we evaluated the in silico interaction of human CXCR4 with human Ub, finding that the Kd at 37 °C was 0.176 nM (data not shown), a much lower value than the interaction of dog CXCR4 with human Ub. These results suggest that the binding affinity of human Ub to human CXCR4 is much higher than that of dog CXCR4.

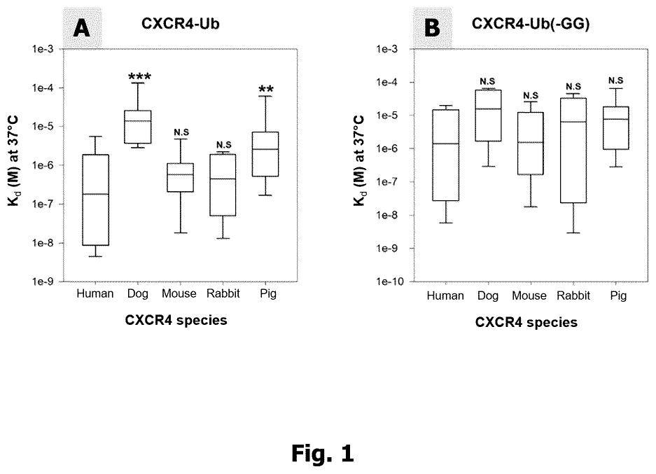

In FIG. 1, the dissociation constant (Kd) values at 37 °C for the binding of ubiquitins Ub (1A) and Ub(-GG) (1B) to the CXCR4 receptor of human, dog, mouse, rabbit, and pig are presented, where, **p<0.01M; ***p<0.001; N.S.=No Significance. The Kruskal-Wallis test was used for statistical analysis (data did not follow a normal distribution).

### Example 2. Use of the Veterinary Peptide Composition in the Treatment of Canine Parvovirus Type II Infection (CPV-2)

In this example, the effect of the veterinary peptide composition is evaluated in 18 dogs infected with canine parvovirus type II (CPV-2). The dogs were randomly assigned to two different groups, wherein the control group (CT+V) received the vehicle, injectable water, and the other group (CT+C) received the veterinary peptide composition (0.5 mg in 1 mL of injectable water, daily). Treatments were administered subcutaneously in the interscapular region every 24 hours for 5 days. The age, sex, breed, and vaccination status of all dogs, as well as the treatment received, are described in Table 1.

TABLE 1

| Group | Dog number | Age (months) | Gender | Breed | Vaccination Status |
|---|---|---|---|---|---|
| CT + V | 1 | 4 | Male | Mixed | No |
| | 2 | 2 | Male | Pitbull | No |
| | 3 | 2 | Male | Chihuahua | No |
| | 4 | 4 | Male | Bloodhound | No |
| | 5 | 4 | Male | Schnauzer | No |
| | 6 | 3 | Male | Mixed | No |
| | 7 | 3 | Male | Poodle | Yes |
| CT + C | 1 | 3 | Female | Mixed | No |
| | 2 | 2 | Female | Mixed | No |
| | 3 | 4 | Male | Mixed | Yes |
| | 4 | 2 | Female | Pomeranian | No |
| | 5 | 5 | Male | Schnauzer | No |
| | 6 | 4 | Female | Pitbull | No |
| | 7 | 3 | Female | Mixed | No |
| | 8 | 5 | Male | Mixed | No |
| | 9 | 4 | Female | Schnauzer | Yes |
| | 10 | 4 | Male | Schnauzer | Yes |
| | 11 | 5 | Male | Mixed | No |

The age (months) of dogs in the Conventional Treatment group (CT+V) was 3.1±0.9, and in the Conventional Treatment/Peptide Veterinary Composition (CT+C) group was 3.7±1.1. Statistical analysis showed no age differences between the groups. The study population consisted of 45% mixed-breed dogs and 55% pure-bred dogs. Only 4/18 dogs had vaccination history (live attenuated virus) at two months of age in the analyzed sample. The drugs administered to the CT+V group of dogs infected with CPV-2 are listed in Table 2. Treatments were classified according to their mechanism of action. The number of dogs receiving each treatment is specified.

TABLE 2

| Treatment | | | | |
|---|---|---|---|---|
| Classification | CT + V | | CT + C | |
| Fluid Therapy | 0.9% NaCl | 7/7 | 0.9% NaCl | 11/11 |
| | Solution | | Solution | |
| Antibiotics | | | Ampicillin | 5/11 |
| | Ampicillin | 3/7 | Metronidazole | 8/11 |
| | Metronidazole | 5/7 | Enrofloxacin | 2/11 |
| | Enrofloxacin | 2/7 | Clindamycin | 1/11 |
| | Cephalexin | 5/7 | Sulfamethoxazole- | 4/11 |
| | | | trimethoprim | |
| Antiemetics | Maropitant citrate | 7/7 | Maropitant citrate | 9/11 |
| | Metoclopramide | 4/7 | Metoclopramide | 5/11 |
| Antiespasmodics | Prifinial | 1/7 | Butilhioscine | 5/11 |
| Gastric mucosal | | | Ranitidine | 8/11 |
| protectants | Ranitidine | 4/7 | Omeprazole | 9/11 |
| | Omeprazole | 7/7 | Sucralfate | 4/11 |
| Analgesics | | | Buprenorphine | 10/11 |
| | Buprenorphine | 7/7 | Lidocaine | 5/11 |
| | Meloxicam | 1/7 | Metamizole | 3/11 |
| Others | 0.5% dextrose | 5/7 | 0.5% dextrose | 8/11 |
| | Aminolite | 2/7 | | |

For the confirmation of CPV-2 infection, two fecal samples were collected using a sterile swab directly from the rectum. The first sample was used as a screening test for rapid parvovirus detection with immunochromatography (Anigen Rapid CPV Ag Test kit, Bionote, South Korea), and the second helped confirm the diagnosis through endpoint PCR wherein the corresponding region (466 bp) was amplified. The sample was placed in a test tube without anticoagulant (BD Vacutainer®, New Jersey, USA) with 1 mL of sterile saline and kept at −20 °C. It was then transferred to a sterile microcentrifuge tube and centrifuged at 12,600×g for 10 minutes. To the obtained supernatant (400-600 μL), DNAzol (Invitrogen®, USA) was added in a 1:1 ratio and kept at −75 °C until the genetic material extraction. In addition, blood samples were taken from the jugular vein on days 0, 1, 2, 3, 4, and 5. The blood was placed in tubes with EDTA anticoagulant (BD Vacutainer®, New Jersey, USA) and kept refrigerated (5±3 °C) until analysis. The quantity obtained (between 1 and 3 mL) was adjusted to the dog's weight per day, ensuring the animal's well-being. Once hematological parameters were evaluated, the samples were centrifuged at 1.233×g for 10 minutes. The obtained plasma was stored at −20 °C until cortisol and neurotransmitters quantification. The samples were obtained between 9 a.m. and 12 p.m.

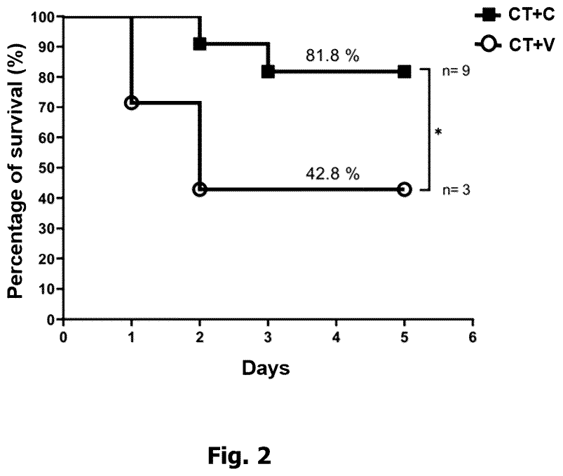

The CT+C group of dogs was administered 0.5 mg of the veterinary peptide composition daily. Survival results are illustrated in FIG. 2, showing a significant increase in the survival rate (%) in dogs co-administered with the veterinary peptide composition (P≤0.043) compared to the CT+V group. The initial number of dogs in the CT+V group was 7, and 4 died. The initial number of dogs in the CT+C group was 11, and only 2 died. The difference in the survival rate percentage was 39.0%. Kaplan-Meier test and Gehan-Breslow-Wilcoxon post-test were performed. *P≤0.05.

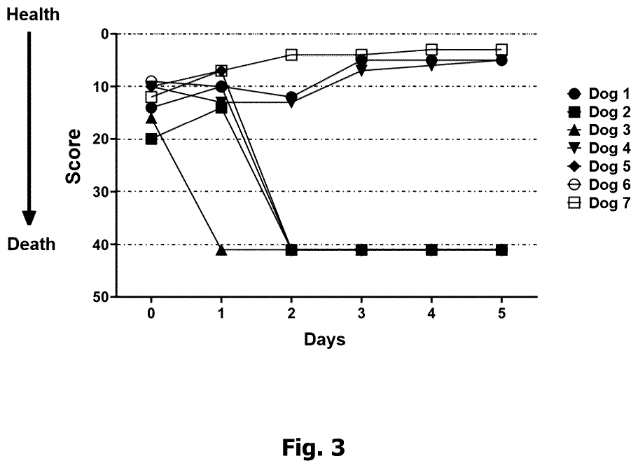

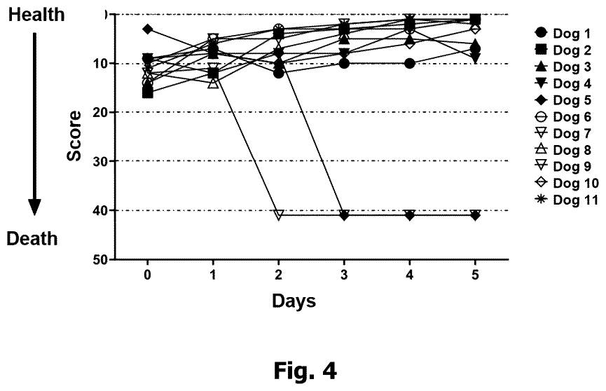

Throughout the trial, a clinical evaluation was conducted to identify whether the veterinary peptide composition could reduce the severity of the clinical condition in dogs infected with canine parvovirus. The evaluation score assigns 0 to healthy dogs, 21 to high severity state, and, if death occurred, an additional value of 20 was added to achieve a score of 41. The Kruskal-Wallis test was performed, and the results are presented in FIGS. 3 and 4, wherein the arrow in each figure indicates that high values (scores from 1 to 41) correlate with the worsening of the dog's clinical condition.

FIG. 3 illustrates the clinical score of the group with Conventional Treatment and vehicle (CT+V) over time per dog. Likewise, FIG. 4 illustrates the clinical score of the group with Conventional Treatment and the veterinary peptide composition (CT+C) over time per dog. Statistical comparison between treatments showed significant differences (H=19.19, df=90.1; P≤0.024), although surviving CT+C dogs tended to have lower scores (healthier) than the CT+V group. The rest of the scores were 8.55±3.32 on day 1 and 6.89±3.33 on day 2 in the CT+C group, while in CT+V, they were 10.00±2.68 on day 1 and 9.67±7.41 on day 2.

Deaths occurred on days 1 and 2 in the CT+V group and on days 2 and 3 in CT+C; subsequently, the scores of the dogs improved to <10 in both groups. This result suggests that conventional treatment plus the veterinary peptide composition induced clinical improvement, reflected in a lower mortality rate compared to CT+V. CT+C dogs who survived the critical period had a better clinical condition. The number of days of hospitalization for surviving dogs was 6.10±2.26 and 7.00±1.00 in the CT+C and CT+V groups, respectively; there was no statistical difference.

Regarding weight between the CT+V and CT+C groups, no significant differences were detected in weight. However, CT+C dogs showed a positive percentage of weight change at all evaluated times, opposite to CT+V dogs, who showed a negative rate of weight change. For example, on the critical day of the disease (day 2), the percentage weight change in the CT+C group was 1.22±4.90, while in the CT+V group, it was −3.06±5.50. At the end of the study, the CT+C group showed a percentage of 1.85±13.61, and the CT+V group was −7.72±9.48. A negative weight change indicates that dogs lost weight compared to their initial value. In this sense, the percentage weight change values indicate that CPV-2 infection leads to weight loss, but the administration of the veterinary peptide composition hinders this loss.

Regarding Hematological Parameters, blood samples were sent to a private veterinary clinical laboratory for a complete blood count. Hematocrit, hemoglobin, and total red and white blood cell counts were evaluated using a CELL-DYN® Emerald hematology analyzer (Abbot, Illinois, USA), which is based on electronic impedance and absorption spectrophotometry. The analyzer was calibrated and verified for the target species. The number of circulating neutrophils and lymphocytes was obtained from a blood smear with a leukocyte differential count. This information was used to obtain the absolute value based on the total leukocyte count. Plasma proteins were identified using a conventional refractometer (RHC-200act, Mexico).

The results show that the CT+C group has fewer circulating neutrophils and lymphocytes on day 2 (critical day) and only neutrophils on day 4 of hospitalization (recovery period) than the CT+V group. The decrease in circulating neutrophils and lymphocytes in the CT+C group is considered to be due to the immunomodulatory activity mediated by the vagal anti-inflammatory pathway. Indirect evidence of immunomodulation is present in the CT+C group if we consider predictive values based on neutrophil and lymphocyte counts in the critical phase of the disease, which suggests a high mortality rate. However, despite this, the CT+C group shows an increased survival rate compared to the CT+V group.

Hematocrit, hemoglobin, and red blood cell values were higher in CT+C than in CT+V. Along with clinical monitoring, the mean values of hematocrit (L/L), hemoglobin (g/L), and total red blood cells (1×10¹²/L) in the CT+C group were 0.34±0.06, 113.90±25.40, and 5.43±1.16, respectively, while in the CT+V group, they were 0.31±0.06, 102.80±18.70, and 5.07±0.93. The concentration of plasma proteins did not show significant differences in the first three days; their values were similar between groups (CT+C=45.73±10.33 g/L vs. CT+V=44.36±9.13 g/L). However, on days 4 and 5, considered recovery days, CT+C showed higher concentrations (day 4=44.67±11.43 g/L; day 5=47.89±14.05 g/L) than CT+V (day 4=35.33±2.52 g/L; day 5=36.33±4.62 g/L).

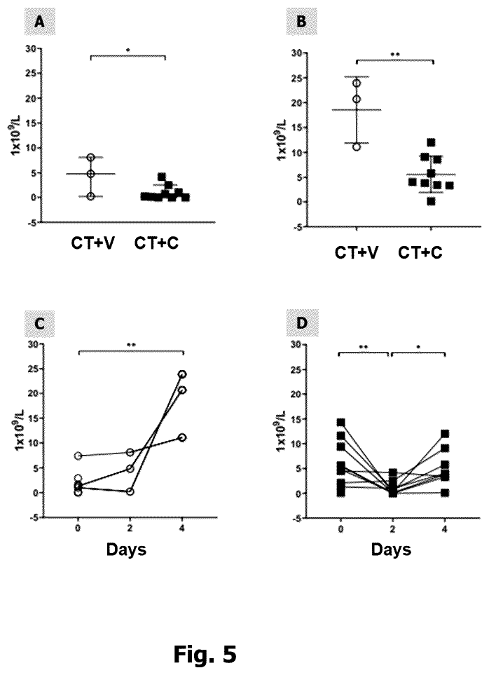

Regarding the influence of the veterinary peptide composition on the concentration of circulating neutrophils, no statistical differences were identified between the groups at the beginning of the trial, but this changed during the trial. First, the concentration of circulating neutrophils on days 0 and 2 was compared between groups, and the CT+C group had a significantly lower neutrophil count than the CT+V group on day 2. Then, the concentration of circulating neutrophils on days 0, 2, and 4 was compared within each group, and CT+V showed a significantly higher neutrophil count on day 4 compared to day 0. Besides, although there were no statistical differences, it is important to note that the neutrophil count was lower on day 2 compared to day 4. On the other hand, a decrease in the concentration of circulating neutrophils in TC+C was observed on the critical day (day 2) compared to days 0 and 4. The results are illustrated in FIG. 5, wherein A is the concentration of circulating neutrophils in surviving dogs on day 2 (critical day); B is the concentration of circulating neutrophils in surviving dogs on day 4 (recovery day); C is the concentration of circulating neutrophils in the CT+V group, and D is the concentration of circulating neutrophils in the CT+C group. Mann-Whitney test was performed. Each point represents a dog. *P<0.05, **P≤0.01.

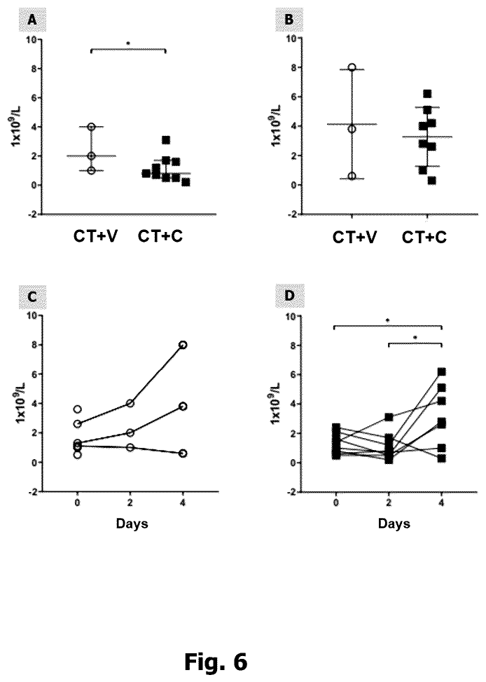

Likewise, the concentration of circulating lymphocytes was compared between the two treatments, showing significant differences on the critical day (day 2); however, no differences were found on the recovery day (day 4). On day 2, the CT+C group exhibited a lower count than CT+V. When comparing days 0, 2, and 4 per treatment, the CT+C group showed a significant increase in circulating lymphocytes during the recovery period vs. days 0 and 2. Although CT+V did not show significant differences, there was an increase in circulating lymphocytes on day 4. The results are illustrated in FIG. 6, wherein A is the concentration of circulating lymphocytes in surviving dogs on day 2 (critical day); B is the concentration of circulating lymphocytes in surviving dogs on day 4 (recovery day); C is the concentration of circulating lymphocytes in the CT+V group, and D is the concentration of circulating lymphocytes in the CT+C group. One-way ANOVA, Tukey's post-hoc test was performed. Each point represents a dog. Mean±SD. *P≤0.05.

Regarding hormonal parameters, the concentration of plasma cortisol was determined in a private veterinary clinical laboratory. Plasma samples were processed on an Architect i2000SR immunoassay analyzer (Abbot, Illinois, USA). The method was based on a heterogeneous chemiluminescent microparticle immunoassay. Statistical analysis showed no differences in cortisol levels between the CT+V and CT+C groups. Despite this, there is a trend towards a decrease in cortisol levels in both groups from day 1. It should be noted that surviving pups from both groups on days 4 and 5 show similar cortisol levels. Cortisol is one of the most used endocrine biomarkers in the management of canine parvovirus. Levels above 224 mmol/L at 48 hours of admission negatively affect dog survival; however, the cortisol cutoff is not a general rule, as observed in our study groups. It is crucial to consider that hyperactivity of the hypothalamic-pituitary-adrenal axis, active in infectious diseases, increases circulating cortisol levels. At elevated and prolonged cortisol concentrations, this hormone can induce immunosuppression and consequently increase mortality. In this example, the cortisol concentration tends to decrease in surviving dogs from both groups, reducing the risk of mortality.

Neurochemical parameters were also determined. The determination was made by extracting norepinephrine, epinephrine, and serotonin from 250 μL of plasma by adding 250 μL of extraction buffer containing 5% ascorbic acid, 200 mM sodium phosphate, 2.5 mM L-cysteine, 2.5 mM EDTA, and 2.4 M perchloric acid. The mixture was incubated at −20 °C for 10 minutes. Supernatants containing norepinephrine, epinephrine, and serotonin were collected after centrifugation at 12,419×g and 4 °C for 10 minutes. They were processed using solid-phase extraction (SPE) with a Hypersep C18 cartridge (Thermo Scientific, Massachusetts, USA) without activation to retain lipids and recover neurotransmitters, and the samples were passed through 0.22 μm filters. Neurotransmitter concentrations were identified using reverse-phase high-performance liquid chromatography (RP-HPLC) in a system consisting of a PU-2089 plus pump (Jasco Inc., Japan) and an X-LC™ 3120FP fluorescence detector (Jasco Inc., Japan). The instruments were controlled using ChromNav software (Jasco Inc., Japan). Chromatographic runs were carried out with a Jupiter C18 column (300 Å, 5μ, 4.6×250 mm, Phanomenex®) at 30 °C. The column was equilibrated with mobile phase A containing 0.1% trifluoroacetic acid in water. A linear gradient from minute 5 to minute 20 was run with mobile phase B containing 0.1% trifluoroacetic acid in acetonitrile. The flow rate was 0.8 mL/min. The fluorescence detector was set to a gain of 1000, attenuation of 32, response of 20 seconds, excitation at 280 nm, and emission at 315 nm. The sample injection volume was 80-100 μL.

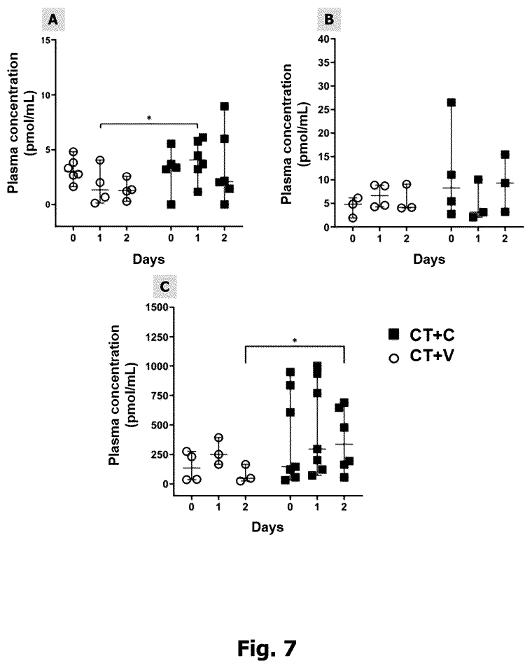

Plasma concentrations of neurotransmitters were evaluated on days 0, 1, and 2, considered the critical period of the disease. The comparison of plasma norepinephrine levels between treatments showed a significant difference on day 1, wherein norepinephrine concentrations were higher in the CT+C group than in the CT+V group during the evaluated period. No differences were detected in plasma epinephrine. The CT+C group showed lower epinephrine values on day 1 than the initial value. Likewise, serotonin values showed a significant increase only in CT+C on day 2 of follow-up (H=12.93, df=60.1; P≤0.035). The results are illustrated in FIG. 7, wherein A is the plasma concentration of norepinephrine; B is the plasma concentration of epinephrine, and C is the plasma concentration of serotonin. Kruskal-Wallis test and Dunn's post-hoc test were performed. Each point represents a dog. Median±5-95% confidence interval. *P≤0.05. CT conventional treatment, I immunomodulator, V vehicle.

In this example, it has been demonstrated that the administration of the veterinary peptide composition as an auxiliary treatment in dogs affected by CPV increases survival. The CT+C group showed an approximately 40% increase in survival compared to conventional treatment. Post-hospital day 2 was selected as the critical period for this study because the highest percentage of mortality (4/6) was observed in the CT+V group. The importance of this period lies in that most prognostic biomarkers are defined between 24 to 48 hours after hospitalization. Mortality on day 1 can be explained by the presence of severe sepsis, metabolic complications, or comorbidities, while dogs that die on the third day are in the period where mortality frequently occurs. Considering that there is no international consensus treatment for canine parvovirus infection, each veterinarian was allowed to apply the required pharmacological treatment to dogs according to their judgment. However, it should be noted that the type of drugs used was similar between experimental groups. In the case of antiemetics, there is evidence that metoclopramide and maropitant have similar effects in dogs infected with CPV-2. Furthermore, it is reported that dogs with CPV-2 have the same outcome in standard hospital and outpatient treatments. In this sense, the positive effects of CT+C were associated merely with the addition of the immunomodulator rather than the pharmacological or clinical treatment where the dogs were treated.

The results shown in this example suggest that the use of the veterinary peptide composition according to the present invention in canine parvovirus infection increases the survival of dogs and improves clinical conditions by promoting the decrease in neutrophil and lymphocyte counts in the critical stage of the disease, reducing plasma cortisol levels, and changes in neurotransmitters levels.

In accordance with the foregoing, it can be observed that the veterinary peptide composition with immunomodulatory effects has been devised not to present variability, unlike other products used in immunotherapy that vary in their peptide content according to their cell origin and manufacturing method, as well as for the integral regulation of the immune system of an animal, and it will be evident to any expert in the field that the modalities of the veterinary composition for immunotherapy as described above and illustrated in the accompanying drawings, are only illustrative but not limiting of this invention. of the present invention. For example, it is possible to use the composition described here in the treatment of different diseases than those presented in the previously described examples. Additionally, the combination of the peptide composition with immunomodulatory effects with other peptides, pharmaceutical and/or immunogenic and/or immunostimulating and/or immunotherapeutic agents is possible, and it can be combined with a physiologically acceptable vehicle for administration.

Therefore, the present invention should not be considered restricted except for what is required by the prior art and by the scope of the appended claims.

1. A veterinary composition, characterized in that it comprises a peptide mixture at a concentration between 33 and 84% (w/v), and at least one veterinary-acceptable adjuvant or vehicle at a concentration of between 16 and 67% (w/v); wherein the peptide mixture includes a complete monomer of human Ubiquitin (Ub), a variant of the human Ubiquitin monomer lacking two terminal glycine (Ub(-GG)), and low molecular weight human peptides.

2. The veterinary composition according to claim 1, further characterized in that it is adapted to be administered to an animal by topical, dermal, transdermal, transmucosal, epidermal, parenteral, gastrointestinal, oral, oropharyngeal, nasopharyngeal and pulmonary routes, including transbronchial and transalveolar.

3. The veterinary composition according to claim 2, further characterized in that the animal is a domestic, exotic, or industrially relevant animal.

4. The veterinary composition according to claim 1, further characterized in that the complete Ub monomer has a molecular mass of approximately 8.56 kDa, and the Ub(-GG) monomer variant has a molecular mass of 8.45 kDa.

5. The veterinary composition according to claim 1, further characterized in that the complete Ub monomer is selected from the amino acid sequence SEQ ID NO:1.

6. The veterinary composition according to claim 1, further characterized in that the Ub(-GG) monomer variant is selected from the amino acid sequence SEQ ID NO:2.

7. The veterinary composition according to claim 1, further characterized in that it comprises 0.168 to 0.268% (w/w) of the complete Ub monomer and the Ub(-GG) monomer variant.

8. The veterinary composition according to claim 1, further characterized in that the average content of the Ub(-GG) monomer variant in the peptide composition with immunomodulatory effects is in a 2:1 ratio with respect to the average content of the complete Ub monomer.

9. The veterinary composition according to claim 1, further characterized in that the low molecular weight human peptides are selected from Ankyrin-1, Protein 4.1, Hemoglobin Alpha Subunit, Hemoglobin Beta Subunit, Hemoglobin Delta Subunit, Complement Protein C3, Calpastatin, Alpha-Synuclein, Fibrinogen Alpha Chain, Cytoplasmic actin 1, Thymosin Beta-4, Thymosin Beta-10, or Zyxin.

10. The veterinary composition according to claim 9, further characterized in that it comprises 0.644 to 0.924% (w/w) of at least one Ankyrin-1 fragment; 0.397 to 0.637% (w/w) of at least one Protein 4.1 fragment; 0.351 to 0.571% (w/w) of at least one Hemoglobin Alpha Subunit fragment; 0.367 to 0.547% (w/w) of at least one Hemoglobin Beta Subunit and/or Hemoglobin Delta Subunit fragment; 0.303 to 0.583% (w/w) of at least one Complement Protein C3 fragment; 0.319 to 0.519% (w/w) of at least one Calpastatin fragment; 0.198 to 0.298% (w/w) of at least one Alpha-Synuclein fragment; 0.151 to 0.311% (w/w) of at least one Fibrinogen Alpha Chain fragment; 0.163 to 0.283% (w/w) of at least one Cytoplasmic actin 1 fragment; 0.12 to 0.2% (w/w) of at least one Thymosin Beta-4 and/or Thymosin Beta-10 fragment; and/or 0.108 to 0.208% (w/w) of at least one Zyxin fragment.

11. The veterinary composition according to claim 9, further characterized in that each low molecular weight human peptide of the human protein Ankyrin-1 corresponds to a fragment of the amino acid sequence SEQ ID NO:3 or SEQ ID NO:4; each low molecular weight human peptide of the human protein Protein 4.1 corresponds to a fragment of the amino acid sequence SEQ ID NO:5; each low molecular weight human peptide of the human protein Hemoglobin Alpha Subunit corresponds to a fragment of the amino acid sequence SEQ ID NO:6 or SEQ ID NO:7; each low molecular weight human peptide of the human protein Hemoglobin Beta Subunit corresponds to a fragment of the amino acid sequence SEQ ID NO:8; each low molecular weight human peptide of the human protein Hemoglobin Delta Subunit corresponds to a fragment of the amino acid sequence SEQ ID NO:9; each low molecular weight human peptide of the human Complement Protein C3 corresponds to a fragment of the amino acid sequence SEQ ID NO:10; each low molecular weight human peptide of the human protein Calpastatin corresponds to a fragment of the amino acid sequence SEQ ID NO:11, SEQ ID NO:12, or SEQ ID NO:13; each low molecular weight human peptide of the human protein Alpha-Synuclein corresponds to a fragment of the amino acid sequence SEQ ID NO:14 or SEQ ID NO:15; each low molecular weight human peptide of the human protein Fibrinogen Alpha Chain corresponds to a fragment of the amino acid sequence SEQ ID NO:16; each low molecular weight human peptide of the human protein Cytoplasmic actin 1 corresponds to a fragment of the amino acid sequence SEQ ID NO:17, SEQ ID NO:18, or SEQ ID NO:19; each low molecular weight human peptide of the human protein Thymosin Beta-4 corresponds to a fragment of the amino acid sequence SEQ ID NO:20; each low molecular weight human peptide of the human protein Thymosin Beta-10 corresponds to a fragment of the amino acid sequence SEQ ID NO:21; and each low molecular weight human peptide of the human protein Zyxin corresponds to a fragment of the amino acid sequence SEQ ID NO:22.

12. The veterinary composition according to claim 1, further characterized in that the veterinary-acceptable adjuvant is a pharmaceutical, and/or immunogenic, and/or immunostimulatory, and/or immunotherapeutic agent.

13. The veterinary composition according to claim 1, further characterized in that the vehicle is a physiologically acceptable vehicle.

14. A veterinary composition for use in the treatment, control, and prevention of a disease in an animal, wherein the veterinary composition comprises a peptide mixture at a concentration between 33 and 84% (w/v), and at least one veterinary-acceptable adjuvant or vehicle at a concentration of between 16 and 67% (w/v); wherein the peptide mixture includes a complete monomer of human Ubiquitin (Ub), a variant of the human Ubiquitin monomer lacking two terminal glycine (Ub(-GG)), and low molecular weight human peptides.

15. The veterinary composition according to claim 14, wherein the disease is related to immunodeficiencies, autoimmune disorders, allergies, or a combination thereof.

16. The veterinary composition according to claim 14, wherein the disease is related to a viral, bacterial, parasitic infection, or a combination thereof.

17. The veterinary composition according to claim 14, wherein the animal is a domestic, exotic, or industrially relevant animal.

18. The veterinary composition according to claim 14, wherein the complete Ub monomer has a molecular mass of approximately 8.56 kDa, and the Ub(-GG) monomer variant has a molecular mass of 8.45 kDa.

19. The veterinary composition according to claim 14, wherein the complete Ub monomer is selected from the amino acid sequence SEQ ID NO:1.

20. The veterinary composition according to claim 14, wherein the Ub(-GG) monomer variant is selected from the amino acid sequence SEQ ID NO:2.

21. The veterinary composition according to claim 14, wherein the composition comprises 0.168 to 0.268% (w/w) of the complete Ub monomer and the Ub(-GG) monomer variant.

22. The veterinary composition for use according to claim 14, wherein the average content of the Ub(-GG) monomer variant in the peptide veterinary composition with immunomodulatory effects is in a 2:1 ratio with respect to the average content of the complete Ub monomer.

23. The veterinary composition for use according to claim 14, wherein the low molecular weight human peptides are selected from Ankyrin-1, Protein 4.1, Hemoglobin Alpha Subunit, Hemoglobin Beta Subunit, Hemoglobin Delta Subunit, Complement Protein C3, Calpastatin, Alpha-Synuclein, Fibrinogen Alpha Chain, Cytoplasmic actin 1, Thymosin Beta-4, Thymosin Beta-10, or Zyxin.

24. The veterinary composition according to claim 23, wherein the composition additionally comprises 0.644 to 0.924% (w/w) of at least one Ankyrin-1 fragment; 0.397 to 0.637% (w/w) of at least one Protein 4.1 fragment; 0.351 to 0.571% (w/w) of at least one Hemoglobin Alpha Subunit fragment; 0.367 to 0.547% (w/w) of at least one Hemoglobin Beta Subunit and/or Hemoglobin Delta Subunit fragment; 0.303 to 0.583% (w/w) of at least one Complement Protein C3 fragment; 0.319 to 0.519% (w/w) of at least one Calpastatin fragment; 0.198 to 0.298% (w/w) of at least one Alpha-Synuclein fragment; 0.151 to 0.311% (w/w) of at least one Fibrinogen Alpha Chain fragment; 0.163 to 0.283% (w/w) of at least one Cytoplasmic actin 1 fragment; 0.12 to 0.2% (w/w) of at least one Thymosin Beta-4 and/or Thymosin Beta-10 fragment; and/or 0.108 to 0.208% (w/w) of at least one Zyxin fragment.

25. The veterinary composition for use according to claim 23, wherein each low molecular weight human peptide of the human Ankyrin-1 protein corresponds to a fragment of the amino acid sequence SEQ ID NO:3 or SEQ ID NO:4; each low molecular weight human peptide of the human Protein 4.1 corresponds to a fragment of the amino acid sequence SEQ ID NO:5; each low molecular weight human peptide of the human Hemoglobin Alpha Subunit corresponds to a fragment of the amino acid sequence SEQ ID NO:6 or SEQ ID NO:7; each low molecular weight human peptide of the human Hemoglobin Beta Subunit corresponds to a fragment of the amino acid sequence SEQ ID NO:8; each low molecular weight human peptide of the human Hemoglobin Delta Subunit corresponds to a fragment of the amino acid sequence SEQ ID NO:9; each low molecular weight human peptide of the human Complement Protein C3 corresponds to a fragment of the amino acid sequence SEQ ID NO:10; each low molecular weight human peptide of the human Calpastatin corresponds to a fragment of the amino acid sequence SEQ ID NO:11, SEQ ID NO:12, or SEQ ID NO:13; each low molecular weight human peptide of the human Alpha-Synuclein corresponds to a fragment of the amino acid sequence SEQ ID NO:14 or SEQ ID NO:15; each low molecular weight human peptide of the human Fibrinogen Alpha Chain corresponds to a fragment of the amino acid sequence SEQ ID NO:16; each low molecular weight human peptide of the human Cytoplasmic actin 1 corresponds to a fragment of the amino acid sequence SEQ ID NO:17, SEQ ID NO:18, or SEQ ID NO:19; each low molecular weight human peptide of the human Thymosin Beta-4 corresponds to a fragment of the amino acid sequence SEQ ID NO:20; each low molecular weight human peptide of the human Thymosin Beta-10 corresponds to a fragment of the amino acid sequence SEQ ID NO:21; and each low molecular weight human peptide of the human Zyxin corresponds to a fragment of the amino acid sequence SEQ ID NO:22.

26. The veterinary composition for use according to claim 14, wherein the veterinary acceptable adjuvant is a pharmaceutical, and/or immunogenic, and/or immunostimulatory, and/or immunotherapeutic agent.

27. The veterinary composition for use according to claim 14, wherein the vehicle is a physiologically acceptable vehicle.

28. The veterinary composition according to claim 14, wherein the composition is adapted to be administered to an animal by topical, dermal, transdermal, transmucosal, epidermal, parenteral, gastrointestinal, oral, oropharyngeal, nasopharyngeal and pulmonary routes, including transbronchial and transalveolar.

29. Use of a veterinary composition for the manufacture of a medicine for the treatment, control, and prevention of a disease in an animal, wherein the veterinary composition comprises a peptide mixture at a concentration between 33 and 84% (w/v), and at least one veterinary-acceptable adjuvant or vehicle at a concentration of between 16 and 67% (w/v); wherein the peptide mixture includes a complete monomer of human Ubiquitin (Ub), a variant of the human Ubiquitin monomer lacking two terminal glycine (Ub(-GG)), and low molecular weight human peptides.

30. The use of a veterinary composition according to claim 29, wherein the disease is related to immunodeficiencies, autoimmune disorders, allergies, or a combination thereof.

31. The use of a veterinary composition according to claim 29, wherein the disease is related to a viral, bacterial, parasitic infection, or a combination thereof.

32. The use of a veterinary composition as stated in claim 29, wherein the animal is a domestic, exotic, or industrially relevant animal.

33. The use of a veterinary composition according to claim 29, wherein the complete Ub monomer has a molecular mass of around 8.56 kDa and the Ub(-GG) monomer variant has a molecular mass of 8.45 kDa.

34. The use of a veterinary composition according to claim 29, wherein the complete Ub monomer is selected from the amino acid sequence SEQ ID NO:1.

35. The use of a veterinary composition according to claim 29, wherein the variant of the Ub(-GG) monomer is selected from the amino acid sequence SEQ ID NO:2.

36. The use of a veterinary composition according to claim 29, wherein the composition comprises 0.168 to 0.268% (w/w) of the complete Ub monomer and the variant of the Ub(-GG) monomer.

37. The use of a veterinary composition according to claim 29, wherein the average content of the Ub(-GG) monomer in the veterinary peptide composition with immunomodulatory effects is in a 2:1 ratio with respect to the average content of the complete Ub monomer.

38. The use of a veterinary composition according to claim 29, wherein the low molecular weight human peptides are selected from Ankyrin-1, Protein 4.1, Hemoglobin Alpha Subunit, Hemoglobin Beta Subunit, Hemoglobin Delta Subunit, Complement Protein C3, Calpastatin, Alpha-Synuclein, Fibrinogen Alpha Chain, Cytoplasmic actin 1, Thymosin Beta-4, Thymosin Beta-10, or Zyxin.

39. The use of a veterinary composition according to claim 38, wherein the composition additionally comprises 0.644 to 0.924% (w/w) of at least one fragment of Ankyrin-1; 0.397 to 0.637% (w/w) of at least one fragment of Protein 4.1; 0.351 to 0.571% (w/w) of at least one fragment of Hemoglobin Alpha Subunit; 0.367 to 0.547% (w/w) of at least one fragment of Hemoglobin Beta Subunit and/or Hemoglobin Delta Subunit; 0.303 to 0.583% (w/w) of at least one fragment of Complement Protein C3; 0.319 to 0.519% (w/w) of at least one fragment of Calpastatin; 0.198 to 0.298% (w/w) of at least one fragment of Alpha-Synuclein; 0.151 to 0.311% (w/w) of at least one fragment of Fibrinogen Alpha Chain; 0.163 to 0.283% (w/w) of at least one fragment of Cytoplasmic actin 1; 0.12 to 0.2% (w/w) of at least one fragment of Thymosin Beta-4 and/or Thymosin Beta-10; and/or 0.108 to 0.208% (w/w) of at least one fragment of Zyxin.

40. The use of a veterinary peptide composition according to claim 38, wherein each low molecular weight human peptide of the human Ankyrin-1 protein corresponds to a fragment of the amino acid sequence SEQ ID NO:3 or SEQ ID NO:4; each low molecular weight human peptide of the human protein Protein 4.1 corresponds to a fragment of the amino acid sequence SEQ ID NO:5; each low molecular weight human peptide of the human Hemoglobin Alpha Subunit protein corresponds to a fragment of the amino acid sequence SEQ ID NO:6 or SEQ ID NO:7; each low molecular weight human peptide of the human Hemoglobin Beta Subunit protein corresponds to a fragment of the amino acid sequence SEQ ID NO:8; each low molecular weight human peptide of the human Hemoglobin Delta Subunit protein corresponds to a fragment of the amino acid sequence SEQ ID NO:9; each low molecular weight human peptide of the human Complement C3 protein corresponds to a fragment of the amino acid sequence SEQ ID NO:10; each low molecular weight human peptide of the human Calpastatin protein corresponds to a fragment of the amino acid sequence SEQ ID NO:11, SEQ ID NO:12 or SEQ ID NO:13; each low molecular weight human peptide of the human Alpha-Synuclein protein corresponds to a fragment of the amino acid sequence SEQ ID NO:14 or SEQ ID NO:15; each low molecular weight human peptide of the human Fibrinogen Alpha Chain protein corresponds to a fragment of the amino acid sequence SEQ ID NO:16; each low molecular weight human peptide of the human Cytoplasmic Actin 1 protein corresponds to a fragment of the amino acid sequence SEQ ID NO:17, SEQ ID NO:18 or SEQ ID NO:19; each low molecular weight human peptide of the human protein Thymosin Beta-4 corresponds to a fragment of the amino acid sequence SEQ ID NO:20; each low molecular weight human peptide of the human protein Thymosin Beta-10 corresponds to a fragment of the amino acid sequence SEQ ID NO:21; and each low molecular weight human peptide of the human protein Zyxin corresponds to a fragment of the amino acid sequence SEQ ID NO:22.

41. The use of a peptide veterinary composition according to claim 29, wherein the veterinary acceptable adjuvant is a pharmaceutical, and/or immunogenic, and/or immunostimulatory, and/or immunotherapeutic agent.

42. The use of a peptide veterinary composition according to claim 29, wherein the veterinary-acceptable vehicle is a physiologically acceptable vehicle.

43. The use of a peptide veterinary composition according to claim 29, wherein the composition is adapted to be administered to an animal by topical, dermal, transdermal, transmucosal, epidermal, parenteral, gastrointestinal, oral, oropharyngeal, nasopharyngeal and pulmonary routes, including transbronchial and transalveolar.


SEQUENCE LISTING (22 sequences)

SEQ ID NO: 1 - protein, 76 aa, Homo sapiens [sequence omitted]

SEQ ID NO: 2 - protein, 74 aa, Homo sapiens
MQIFVKTLTGKTITLEVEPSDTIENVKAKIQDKEGIPPDQQRLIFAGKQLEDGRTLSDYNIQKESTLHLVLRLR

SEQ ID NO: 3 - protein, 1881 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVTVEGPLEDPSELEVDIDYFMKHSKDHTSTPNP

SEQ ID NO: 4 - protein, 131 aa, Homo sapiens
MWTFVTQLLVTLVLLSFFLVSCQNVMHIVRGSLCFVLKHIHQELDKELGESEGLSDDEETISTRVVRRRVFLKGNEFQNIPGEQVTEEQFTDEQGNIVTKKIIRKVVRQIDLSSADAAQEHEEDHTSTPNP

SEQ ID NO: 5 - protein, 864 aa, Homo sapiens
MTTEKSLVTEAENSQHQQKEEGEEAINSGQQEPQQEESCQTAAEGDNWCEQKLKASNGDTPTHEDLTKNKERTSESRGLSRLFSSFLKRPKSQVSEEEGKEVESDKEKGEGGQKEIEFGTSLDEEIILKAPIAAPEPELKTDPSLDLHSLSSAETQPAQEELREDPDFEIKEGEGLEECSKIEVKEESPQSKAETELKASQKPIRKHRNMHCKVSLLDDTVYECVVEKHAKGQDLLKRVCEHLNLLEEDYFGLAIWDNATSKTWLDSAKEIKKQVRGVPWNFTFNVKFYPPDPAQLTEDITRYYLCLQLRQDIVAGRLPCSFATLALLGSYTIQSELGDYDPELHGVDYVSDFKLAPNQTKELEEKVMELHKSYRSMTPAQADLEFLENAKKLSMYGVDLHKAKDLEGVDIILGVCSSGLLVYKDKLRINRFPWPKVLKISYKRSSFFIKIRPGEQEQYESTIGFKLPSYRAAKKLWKVCVEHHTFFRLTSTDTIPKSKFLALGSKFRYSGRTQAQTRQASALIDRPAPHFERTASKRASRSLDGAAAVDSADRSPRPTSAPAITQGQVAEGGVLDASAKKTVVPKAQKETVKAEVKKEDEPPEQAEPEPTEAWKVEKTHIEVTVPTSNGDQTQKLAEKTEDLIRMRKKKRERLDGENIYIRHSNLMLEDLDKSQEEIKKHHASISELKKNFMESVPEPRPSEWDKRLSTHSPFRTLNINGQIPTGEGPPLVKTQTVTISDNANAVKSEIPTKDVPIVHTETKTITYEAAQTDDNSGDLDPGVLLTAQTITSETPSSTTTTQITKTVKGGISETRIEKRIVITGDADIDHDQVLVQAIKEAKEQHPDMSVTKVVVHQETEIADE

SEQ ID NO: 6 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 7 - protein, 110 aa, Homo sapiens
MFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 8 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 9 - protein, 104 aa, Homo sapiens
MVHLTPEEKTAVNALWGKVNVDAVGGEALGRLLVVYPWTQRFFESFGDLSSPDAVMGNPKVKAHGKKVLGAFSDGLAHLDNLKGTFSQLSELHCDKLHVDPENF

SEQ ID NO: 10 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 11 - protein, 708 aa, Homo sapiens
MNPTETKAIPVSQQMEGPHLPNKKKHKKQAVKTEPEKKSQSTKLSVVHEKKSQEGKPKEHTEPKSLPKQASDTGSNDAHNKKAVSRSAEQQPSEKSTEPKTKPQDMISAGGESVAGITAISGKPGDKKKEKKSLTPAVPVESKPDKPSGKSGMDAALDDLIDTLGGPEETEEENTTYTGPEVSDPMSSTYIEELGKREVTIPPKYRELLAKKEGITGPPADSSKPIGPDDAIDALSSDFTCGSPTAAGKKTEKEESTEVLKAQSAGTVRSAAPPQEKKRKVEKDTMSDQALEALSASLGTRQAEPELDLRSIKEVDEAKAKEEKLEKCGEDDETIPSEYRLKPATDKDGKPLLPEPEEKPKPRSESELIDELSEDFDRSECKEKPSKPTEKTEESKAAAPAPVSEAVCRTSMCSIQSAPPEPATLKGTVPDDAVEALADSLGKKEADPEDGKPVMDKVKEKAKEEDREKLGEKEETIPPDYRLEEVKDKDGKPLLPKESKEQLPPMSEDFLLDALSEDFSGPQNASSLKFEDAKLAAAISEVVSQTPASTTQAGAPPRDTSQSDKDLDDALDKLSDSLGQRQPDPDENKPMEDKVKEKAKAEHRDKLGERDDTIPPEYRHLLDDNGQDKPVKPPTKKSEDSKKPADDQDPIDALSGDLDSCPSTTETSQNTAKDKCKKAASSSKAPKNGGKAKDSAKTTEETSKPKDD

SEQ ID NO: 12 - protein, 430 aa, Homo sapiens
MGQFLSSTFLEGSPATVWHDKLCDGERRGAREAVRIFQDQAKAKEEKLEKCGEDDETIPSEYRLKPATDKDGKPLLPEPEEKPKPRSESELIDELSEDFDRSECKEKPSKPTEKTEESKAAAPAPVSEAVCRTSMCSIQSAPPEPATLKGTVPDDAVEALADSLGKKEADPEDGKPVMDKVKEKAKEEDREKLGEKEETIPPDYRLEEVKDKDGKPLLPKESKEQLPPMSEDFLLDALSEDFSGPQNASSLKFEDAKLAAAISEVVSQTPASTTQAGAPPRDTSSDKDLDDALDKLSDSLGQRQPDPDENKPMEDKVKEKAKAEHRDKLGERDDTIPPEYRHLLDDNGQDKPVKPPTKKSEDSKKPADDQDPIDALSGDLDSCPSTTETSQNTAKDKCKKAASSSKAPKNGGKAKDSAKTTEETSKPKDD

SEQ ID NO: 13 - protein, 91 aa, Homo sapiens
MSDQALEALSASLGTRQAEPELDLRSIKEVDEDKDGKPLLPEPEEKPKPRSESELIDELSEDFDRSECKEKPSKPTEKTEESKAAAPAPVS

SEQ ID NO: 14 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 15 - protein, 98 aa, Homo sapiens
MDVFMKGLSKAKEGVVAAAEKTKQGVAEAAGKTKEGVLYVVAEKTKEQVTNVGGAVVTGVTAVAQKTVEGAGSIAAATGFVKKDQLGKEGYQDYEPEA

SEQ ID NO: 16 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 17 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 18 - protein, 164 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVP

SEQ ID NO: 19 - protein, 1075 aa, Homo sapiens
MVVEVDSMPAASSVKKPFGLRSKMGKWCCRCFPCYRESGKSNVGTSGDHDDSAMKTLRSKMGKWCHHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPEQELKLTSEEESQRFKGSENSQPEKMSQELEINKDGDREVEEEMKKHESNNVGLLENLTNGVTAGNGDNGLIPQRKSRTPENQQFPDNESEEYHRICELLSDYKEKQMPKYSSENSNPEQDLKLTSEEESQRLKGSENGQPEKRSQEPEINKDGDRELENFMAIEEMKKHGSTHVGFPENLTNGATAGNGDDGLIPPRKSRTPESQQFPDTENEEYHSDEQNDTQKQFCEEQNTGILHDEILIHEEKQIEVVEKMNSELSLSCKKEKDVLHENSTLREEIAMLRLELDTMKHQSQLREKKYLEDIESVKKKNDNLLKALQLNELTMDDDTAVLVIDNGSGMCKAGFAGDDAPRAVFPSIVGRPRQQGMMGGMHQKESYVGKEAQSKRGILTLKYPMEHGIITNWDDMEKIWHHTFYNELRVAPEEHPILLTEAPLNPKANREKMTQIMFETFNTPAMYVAIQAVPSLYTSGRTTGIVMDSGDGVTHTVPIYEGNALPHATLRLDLAGRELPDYLMKILTERGYRFTTMAEREIVRDIKEKLCYVALDFEQEMATAASSSSLEKSYELPDGQVITIGNERFRCPEALFQPCFLGMESCGIHETTFNSIMKSDVDIRKDLYTNTVLSGGTTMYPGMAHRMQKEIAALAPSMMKIRIIAPPKRKYSVWVGGSILASLSTFQQMWISKQEYDESGPSIVHRKCF

SEQ ID NO: 20 - protein, 44 aa, Homo sapiens
MSDKPDMAEIEKFDKSKLKKTETQEKNPLPSKETIEQEKQAGES

SEQ ID NO: 21 - protein, 44 aa, Homo sapiens
MADKPDMGEIASFDKAKLKKTETQEKNTLPTKETIEQEKRSEIS

SEQ ID NO: 22 - protein, 572 aa, Homo sapiens
MAAPRPSPAISVSVSAPAFYAPQKKFGPVVAPKPKVNPFRPGDSEPPPAPGAQRAQMGRVGEIPPPPPEDFPLPPPPLAGDGDDAEGALGGAFPPPPPPIEESFPPAPLEEEIFPSPPPPPEEEGGPEAPIPPPPQPREKVSSIDLEIDSLSSLLDDMTKNDPFKARVSSGYVPPPVATPFSSKSSTKPAAGGTAPLPPWKSPSSSQPLPQVPAPAQSQTQFHVQPQPQPKPQVQLHVQSQTQPVSLANTQPRGPPASSPAPAPKFSPVTPKFTPVASKFSPGAPGGSGSQPNQKLGHPEALSAGTGSPQPPSFTYAQQREKPRVQEKQHPVPPPAQNQNQVRSPGAPGPLTLKEVEELEQLTQQLMQDMEHPQRQNVAVNELCGRCHQPLARAQPAVRALGQLFHIACFTCHQCAQQLQGQQFYSLEGAPYCEGCYTDTLEKCNTCGEPITDRMLRATGKAYHPHCFTCVVCARPLEGTSFIVDQANRPHCVPDYHKQYAPRCSVCSEPIMPEPGRDETVRVVALDKNFHMKCYKCEDCGKPLSIEADDNGCFPLDGHVLCRKCHTARAQT